(19) United States
(12) Patent Application Publication
(10) Pub. No.: US 2011/0245351 A1
(43) Pub. Date: Oct. 6, 2011

(54) MEDICAL ADHESIVE FOR SURGERY

(75) Inventors: Heike Heckroth, Odenthal (DE); Burkhard Köhler, Zierenberg (DE)

(73) Assignee: BAYER MATERIALSCIENCE AG, Leverkusen (DE)

(21) Appl. No.: 13/133,691

(22) PCT Filed: Nov. 28, 2009
(86) PCT No.: PCT/EP2009/008498

§ 371 Date: Jun. 9, 2011

(30) Foreign Application Priority Data
Dec. 12, 2008 (EP) 08021576.7

(51) Int. Cl. A61K 47/34; A61P 17/02; C12N 5/02; C12N 5/071; C08G 18/34; B67D 7/06

(52) U.S. Cl. 514/772.3; 435/325; 435/366; 523/105; 528/84; 222/129

(57) ABSTRACT

The invention relates to novel rapidly-curing adhesives made from hydrophilic polyisocyanate prepolymers for application in surgery.

The present invention relates to novel, rapidly curing adhesives based on hydrophilic polyisocyanate prepolymers for use in surgery.

In recent years, growing interest has developed in the replacement or reinforcement of surgical sutures through the use of suitable adhesives. Particularly in the field of plastic surgery, in which emphasis is placed on thin and, as far as is possible, invisible scars, adhesives are increasingly being used.

Tissue adhesives must have a number of properties in order to be accepted by surgeons as a substitute for sutures. These include easy workability and an initial viscosity such that the adhesive cannot penetrate or drain into deeper tissue layers. In conventional surgery, rapid curing is required, whereas in plastic surgery correction of the adhesive suture should be possible and hence the rate of curing must not be too rapid (ca. 1-5 mins). The adhesive layer should be a flexible, transparent film, which is not degraded in a period of less than three weeks. The adhesive must be biocompatible and must have neither histotoxicity nor thrombogenicity nor any allergenic potential.

Various materials which are used as tissue adhesives are commercially available. These include the cyanoacrylates Dermabond® (2-octyl cyanoacrylate) and Histoacryl Blue® (butyl cyanoacrylate). However, the rapid curing time and the brittleness of the joint limit their use. Owing to their poor biodegradability, cyanoacrylates are only suitable for external surgical sutures.

As alternatives to the cyanoacrylates, biological adhesives such as peptide-based substances (BioGlue®) or fibrin adhesives (Tissucol) are available. Apart from the high cost, fibrin adhesives are characterized by relatively weak adhesive strength and rapid degradation, such that they can only be used for smaller incisions on unstretched skin.

Isocyanate-containing adhesives are all based on an aromatic diisocyanate and a hydrophilic polyol, the isocyanates TDI and MDI preferably being used (US 20030135238, US 20050129733). Both can bear electron-withdrawing substituents in order to increase the reactivity (WO-A 03/9323).

Hitherto, problems were the low mechanical strength (U.S. Pat. No. 5,156,613), excessively slow curing rate (U.S. Pat. No. 4,806,614), excessively rapid biodegradability (U.S. Pat. No. 6,123,667) and uncontrolled swelling (U.S. Pat. No. 6,265,016).

According to the patent US 20030135238, only polyurethane prepolymers with a trifunctional or branched structure and which are capable of forming hydrogels are suitable adhesives. At the same time, the adhesive must be capable of forming a covalent bond to the tissue. US 20030135238 and US 20050129733 describe the synthesis of trifunctional, ethylene oxide-rich TDI and IPDI (US 20030135238) based prepolymers which react with water or with tissue fluids to give the hydrogel. Hitherto, sufficiently rapid curing was only attained with the use of aromatic isocyanates, which however react with foam formation. This results in penetration of the adhesive into the wound and hence to the pushing apart of the wound borders, which results in poorer healing with increased scarring. In addition, the mechanical strength and the adhesion of the adhesive layer are decreased by the foam formation. Moreover, owing to the high reactivity of the prepolymers, a reaction of the isocyanate residues with the tissue occurs, as a result of which denaturation, recognizable by a white colouration of the tissue, often occurs.

Lysine diisocyanate has been studied as a replacement for the aromatic isocyanates, but because of its low reactivity this reacts only slowly or not at all with tissue (US 20030135238).

Aliphatic isocyanates have been fluorinated in order to increase the reactivity (U.S. Pat. No. 5,173,301), but this resulted in spontaneous self-polymerisation of the isocyanate.

EP-A 0 482 467 describes the synthesis of a surgical adhesive based on an aliphatic isocyanate (preferably HDI) and a polyethylene glycol (Carbowax 400). Curing takes place on addition of 80-100% water and a metal carboxylate (potassium octoate) as catalyst, during which a foam forms, which is stabilised with silicone oil.

Systems based on aliphatic isocyanates display only inadequate reactivity and hence an excessively slow curing time. Admittedly, the reaction rate could be increased by the use of metal catalysts, as described in EP-A 0 482 467, but foam formation occurred, with the problems described above.

The combination of aspartic acid esters for the crosslinking of prepolymers with the formation of a strong tissue adhesive is already described in the non-prepublished European patent applications Nos. 08012901.8, 08004134.6, 08001290.9 and 07012984.6. On the other hand, alternative compounds for the amine curing of the prepolymers are not mentioned.

The provision of active substances in tissue adhesives is of interest for various fields. Through the use of analgesics, the sensitivity to pain at the site to be treated is decreased or eliminated, as a result of which a subcutaneous injection of an analgesic can be dispensed with. Particularly in the field of veterinary medicine, in which painkillers are only rarely used for topical incisions such as castrations or mulesing in sheep, an analgesic integrated in the adhesive is indicated. In addition, the risk of traumatic shock is reduced by decreasing the sensitivity to pain.

The use of substances with antimicrobial/antiseptic action prevents penetration of germs into the wound and effects the destruction of any bacteria already present. This is of particular interest in veterinary medicine, since here it is only possible to operate aseptically in rare cases. The same applies for compounds with antimycotic activity for the treatment of fungal infections.

In general, pharmacologically active compounds are understood to mean substances and preparations of substances which are intended for use on or in the human or animal body in order to heal, alleviate, prevent or identify diseases, illnesses, physical injury or pathological symptoms. These also include substances and preparations for protecting against, eliminating or rendering harmless pathogens, parasites or extraneous substances.

A tissue adhesive should:
form a strong bond to the tissue
form a transparent film
form a flexible suture
as a result of controlled viscosity, be easy to apply and not penetrate into deeper tissue layers
have a curing time of a few seconds up to 10 minutes depending on the field of use
exhibit no significant exotherm during curing
be biocompatible and exhibit no cell and tissue toxicity

In the context of the present invention, tissue is understood to mean associations of cells which consist of cells of the same form and function, such as epithelium (skin), epithelial tissue, myocardium, connective or supporting tissue, muscles, nerves and cartilage. Inter alia, this also includes all organs built up of cell associations such as the liver, kidneys, lung, heart, etc.

It has now been found that through a combination of prepolymers with isocyanate groups based on aliphatic isocyanates, such as those in the non-prepublished European patent applications Nos. 08012901.8, 08004134.6, 08001290.9 and 07012984.6 with special secondary diamines structurally derived from amino acids, tissue adhesives can be produced which also fulfil the conditions mentioned above.

The subject of the present invention is therefore adhesive systems comprising

A) prepolymers with isocyanate groups obtainable from
A1) aliphatic isocyanates and
A2) polyols with number average molecular weights of ≧400 g/mol and average OH-group contents of 2 to 6
   B1) secondary diamines of the general formula (I)

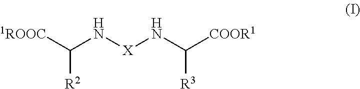

wherein
X is a divalent optionally heteroatom-containing hydrocarbon residue,
R¹ mutually independently are the same or different organic residues which have no Zerevitinov-active hydrogen and

R², R³ mutually independently are optionally substituted and/or heteroatom-containing hydrocarbon residues with 1 to 9 carbon atoms or hydrogen,

B2) optionally organic fillers, which exhibit a viscosity measured according to DIN 53019 at 23° C. in the range from 10 to 6000 mPas and
C) optionally one or more pharmacologically active compounds.

According to a preferred embodiment of the invention, R² and/or R³ have the meaning given above, but are not CH₂—COOR′.

For the definition of Zerevitinov-active hydrogen, reference is made to Römpp Chemie Lexikon, Georg Thieme Verlag Stuttgart. Preferably, groups with Zerevitinov-active hydrogen are understood to mean OH, NH or SH.

The prepolymers with isocyanate groups used in A) are obtainable by reaction of isocyanates with polyols with hydroxy groups optionally with the addition of catalysts and auxiliary agents and additives.

In A1), as isocyanates for example monomeric aliphatic or cycloaliphatic di- or triisocyanates such as 1,4-butylene diisocyanate (BDI), 1,6-hexamethylene diisocyanate (HDI), isophorone diisocyanate (IPDI), 2,2,4- and/or 2,4,4-trimethylhexamethylene diisocyanate, the isomeric bis-(4,4′-isocyanatocyclohexyl)-methane or mixtures thereof of any isomer content, 1,4-cyclo-hexylene diisocyanate, 4-isocyanatomethyl-1,8-octane diisocyanate (nonane triisocyanate), and alkyl 2,6-diisocyanatohexanoate (lysine diisocyanate) with C1-C8 alkyl groups can be used.

As well as the aforesaid monomeric isocyanates, higher molecular weight derivatives thereof with uretdione, isocyanurate, urethane, allophanate, biuret, iminooxadiazine dione or oxadiazine trione structure and mixtures thereof can also be used.

Preferably in A1), isocyanates of the aforesaid type with exclusively aliphatically or cycloaliphatically bound isocyanate groups or mixtures thereof are used.

The isocyanates or isocyanate mixtures used in A1) preferably have an average NCO group content of 2 to 4, particularly preferably 2 to 2.6 and quite especially preferably 2 to 2.4.

In a particularly preferred embodiment, hexamethylene diisocyanate is used in A1).

For constructing the prepolymer in A2), essentially all polyhydroxy compounds with 2 or more OH groups per molecule in themselves known to the person skilled in the art can be used. These can be for example polyester polyols, polyacrylate polyols, polyurethane polyols, polycarbonate polyols, polyether polyols, polyester polyacrylate polyols, polyurethane polyacrylate polyols, polyurethane polyester polyols, polyurethane polyether polyols, polyurethane polycarbonate polyols, polyester polycarbonate polyols or any mixtures thereof with one another.

The polyols used in A2) preferably have an average OH group content of 3 to 4.

Further, the polyols used in A2) preferably have a number average molecular weight of 400 to 20000 g/mol, particularly preferably 2000 to 10000 g/mol and quite especially preferably 4000 to 8500 g/mol.

Polyether polyols are preferably polyalkylene oxide polyethers based on ethylene oxide and optionally propylene oxide.

These polyether polyols are preferably based on starter molecules with two or more functional groups such as alcohols or amines with two or more functional groups.

Examples of such starters are water (regarded as a diol), ethylene glycol, propylene glycol, butylene glycol, glycerine, TMP, sorbitol, pentaerythritol, triethanolamine, ammonia or ethylene-diamine.

Preferred polyalkylene oxide polyethers correspond to those of the aforesaid type and have a content of ethylene oxide-based units of 50 to 100 mol %, preferably of 60 to 90 mol % and quite especially preferably 70 to 80 mol % based on the total quantity of alkylene oxide units contained.

Preferred polyester polyols are the polycondensates, in themselves known, from di- and optionally tri- and tetraols and di- and optionally tri- and tetracarboxylic acids or hydroxycarboxylic acids or lactones. Instead of the free polycarboxylic acids, the corresponding polycarboxylic anhydrides or corresponding polycarboxylate esters of lower alcohols can also be used for the production of the polyesters.

Examples of suitable diols are ethylene glycol, butylene glycol, diethylene glycol, triethylene glycol, polyalkylene glycols such as polyethylene glycol, and also 1,2-propanediol, 1,3-propane-diol, butanediol(1,3), butanediol(1,4), hexanediol(1,6) and isomers, neopentyl glycol or neopentyl glycol hydroxypivalate, among which hexanediol(1,6) and isomers, butanediol(1,4), neopentyl glycol and neopentyl glycol hydroxypivalate are preferred. In addition, polyols such as trimethylolpropane, glycerine, erythritol, pentaerythritol, trimethylolbenzene or trishydroxyethyl isocyanurate can also be used.

As dicarboxylic acids, phthalic acid, isophthalic acid, terephthalic acid, tetrahydrophthalic acid, hexahydrophthalic acid, cyclohexanedicarboxylic acid, adipic acid, azelaic acid, sebacic acid, glutaric acid, tetrachlorophthalic acid, maleic acid, fumaric acid, itaconic acid, malonic acid, suberic acid, 2-methylsuccinic acid, 3,3-diethylglutaric acid and/or 2,2-dimethylsuccinic acid can be used. The corresponding anhydrides can also be used as the acid source.

As long as the average functional group content of the polyol to be esterified is >2, monocarboxylic acids such as benzoic acid and hexanecarboxylic acid can also be used as well.

Preferred acids are aliphatic or aromatic acids of the aforesaid type. Particularly preferable are adipic acid, isophthalic acid and phthalic acid.

Examples of hydroxycarboxylic acids which can be used as reaction participants as well in the production of a polyester polyol with terminal hydroxy groups are hydroxycaproic acid, hydroxybutyric acid, hydroxydecanoic acid, hydroxystearic acid and the like. Suitable lactones are caprolactone, butyrolactone and homologues. Caprolactone is preferred.

Likewise, hydroxy group-containing polycarbonates, preferably polycarbonate diols, with number average molecular weights Mₙ of 400 to 8000 g/mol, preferably 600 to 3000 g/mol, can be used. These are obtainable by reaction of carbonic acid derivatives such as diphenyl carbonate, dimethyl carbonate or phosgene with polyols, preferably diols.

Examples of such diols are ethylene glycol, 1,2- and 1,3-propanediol, 1,3- and 1,4-butanediol, 1,6-hexanediol, 1,8-octanediol, neopentyl glycol, 1,4-bishydroxymethylcyclohexane, 2-methyl-1,3-propanediol, 2,2,4-trimethylpentane-1,3-diol, dipropylene glycol, polypropylene glycols, dibutylene glycol, polybutylene glycols, bisphenol A and lactone-modified diols of the aforesaid type.

Preferably polyether polyols of the aforesaid type are used for constructing the prepolymer.

For the production of the prepolymer, the compounds of the component A1) are reacted with those of the component A2) at an NCO/OH ratio of preferably 4:1 to 12:1, particularly preferably 8:1 and then the content of unreacted compounds of the component A1) is removed by suitable methods. Thin film distillation is normally used for this, products low in residual monomer with residual monomer contents of less than 1 wt. %, preferably less than 0.5 wt. %, quite especially preferably less than 0.1 wt. %, being obtained.

Optionally, stabilisers such as benzoyl chloride, isophthaloyl chloride, dibutyl phosphate, 3-chloropropionic acid or methyl tosylate can be added during the production process.

The reaction temperature here is 20 to 120° C., preferably 60 to 100° C.

X in formula (I) can be a divalent aliphatic or cycloaliphatic hydrocarbon residue, which can bear heteroatoms such as oxygen, sulphur or substituted/unsubstituted nitrogen in the C—C chain. A substitution on the nitrogen can be an alkyl group, preferably methyl, ethyl or propyl. Preferably X in formula (I) is an alkyl chain with 4 to 7 carbon atoms.

R² and R³ are preferably derived from natural amino acids of the general formula R²—CH(NH₂)—COOH or R³—CH(NH₂)—COOH from the group alanine, leucine, valine, t-leucine, isoleucine, phenylalanine, dihydroxyphenylalanine (dopa), tyrosine, histidine, methionine, proline, arginine, asparagine, aspartic acid, cysteine, glutamic acid, glutamine, lysine, serine and threonine.

Particularly preferably here R² and R³ are mutually independently —CH₃, —CH₂CH(CH₃)₂, —CH(CH₃)₂, —C(CH₃)₃, —CH(CH₃)CH₂CH₃, phenyl, 2,3-dihydroxyphenyl, alkyl or cycloalkyl residues with 1 to 9, preferably 1 to 4 C atoms, which optionally have a heteroatom from the group sulphur, oxygen and nitrogen as part of a functional group in the chain or terminally. Terminal hydroxyl, amino and carboxy groups can of course also be alkylated.

Quite especially preferably, R² and R³ are mutually independently —CH₂CH(CH₃)₂, —CH(CH₃)₂, —C(CH₃)₃ or —CH(CH₃)CH₂CH₃.

In principle, R² and R³ can vary mutually independently within the scope of the aforesaid ranges, however, preferably R²═R³.

In principle, the configuration at the stereo centre in the α position to the amino- or R²/R³ group is immaterial for the functioning of the present invention. For the production of the secondary diamines of the formula (I), amino acids or esters thereof are used as starting materials, hence these can in each case be used enantiomerically pure or as racemic mixtures.

R¹ is preferably a C₁ to C₁₀ alkyl residue, particularly preferably methyl or ethyl.

In a preferred embodiment of the invention, R¹=methyl, X being based on 1,5-diaminopentane as the n-valent amine.

The production of the secondary diamines of the component B1) can for example be effected in a known manner by reductive amination of the corresponding oxo acetate with a primary difunctional amine (Equation 1).

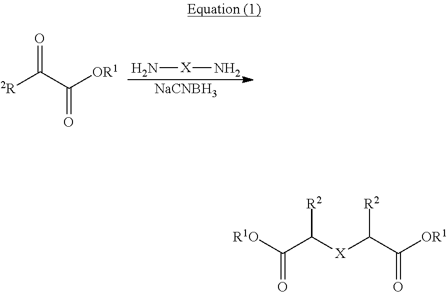

Preferred primary difunctional amities X(NH₂)₂ are ethylenediamine, 1,2-diaminopropane, 1,4-diaminobutane, 1,6-diaminohexane, 2,5-diamino-2,5-dimethylhexane, 2,2,4- and/or 2,4,4-trimethyl-1,6-diaminohexane, 1,11-diaminoundecane, 1,12-diaminododecane, 1-amino-3,3,5-trimethyl-5-aminomethylcyclohexane, 2,4- and/or 2,6-hexahydrotoluoylenediamine, 2,4′- and/or 4,4′-(diaminodicyclohexylmethane, 3,3′-dimethyl-4,4′-diaminodicyclohexylmethane, 2,4,4′-tri-amino-5-methyldicyclohexylmethane and polyether amines with aliphatically bound primary amino groups with a number average molecular weight Mₙ of 148 to 6000 g/mol.

Particularly preferred primary difunctional amines are 1,3-diaminopropane, 1,3-diaminobutane, 1,5-diaminopentane and 1,6-diaminohexane.

The preparation can also be effected by reaction of the protected amino acid ester with the corresponding dialdehyde via the diimine and subsequent deprotection (Equation 2).

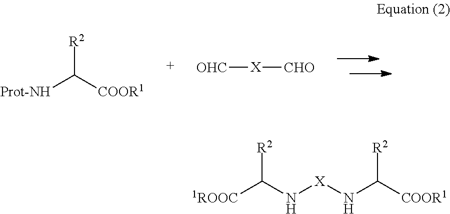

Depending on the chain length x, the products can also be obtained by reaction of the corresponding amino acid ester hydrochloride with a dibromoalkyl compound:

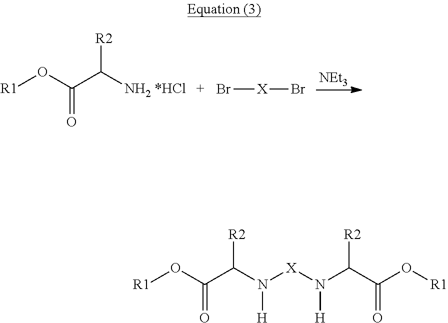

The reaction can also be performed in such a manner that an unsymmetrical end product is formed (Equation 4):

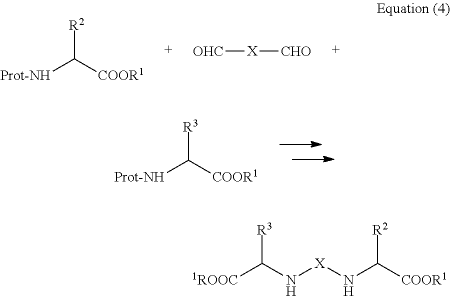

In the Equations (2, 3 and 4) natural and non-natural amino acid esters are used as educts. In order to prevent an intramolecular cyclization, the amino acid esters are N-terminally protected. As protective groups, all suitable systems known to the chemist can be used (e.g. tert.-butoxycarbonyl (Boc) or benzyloxycarbonyl (Z)).

With the use of the dialdehydes or dibromides OHC—X—CHO or Br—X—Br respectively, X can be an alkyl chain with 2 to 6, preferably 2 or 3 carbon atoms.

The organic liquid fillers used in B2) are preferably not cytotoxic according to cytotoxicity measurement as per ISO 10993.

For example liquid polyethylene glycols such as PEG 200 to PEG 600, mono or dialkyl ethers thereof such as PEG 500 dimethyl ether, liquid polyether and polyester polyols, liquid polyesters such as for example Ultramoll (Lanxess AG, Leverkusen, DE) and glycerine and liquid derivatives thereof such as for example triacetin (Lanxess AG, Leverkusen, DE) can be used as organic fillers.

Preferably the organic fillers of the component B2) are compounds with hydroxy groups. Preferred compounds with hydroxy groups are polyether and/or polyester polyols, particularly preferably polyether polyols.

The preferred organic fillers of the component B2) preferably have average OH group contents of 1.5 to 3, particularly preferably 1.8 to 2.2, quite especially preferably 2,0.

The preferred organic fillers of the component B2) preferably have repeating units derived from ethylene oxide.

The viscosity of the organic fillers of the component B2) is preferably 50 to 4000 mPas at 23° C. measured as per DIN 53019.

In a preferred embodiment of the invention, polyethylene glycols are used as organic fillers of the component B2). These preferably have a number average molecular weight of 100 to 1000 g/mol, particularly preferably 200 to 400 g/mol.

The weight ratio of B1) to B2) is 1:0 to 1:20, preferably 1:0 to 1:12.

The weight ratio of the component B2) based on the total quantity of the mixture of B1, B2 and A lies in the range from 0 to 100%, preferably 0 to 60%.

Pharmacologically active substances can inter alia, but not exclusively be:
a) Analgesics with and without anti-inflammatory action
b) Anti-inflammatories
c) Substances with antimicrobial activity
d) Antimycotics
e) Substances with antiparasitic activity

The active substance is preferably soluble in the curing component B1) at room temperature, but can also be used suspended in B1). In a preferred embodiment of the invention, the active substance is dissolved or suspended in a mixture of curing component B1) and filler B2), polyethylene glycols with a number average molecular weight of 100 to 1000 g/mol, particularly preferably 200 to 400 g/mol preferably being used as B2).

The concentration of the active substance added is based on the therapeutically necessary doses and is about 0.001 wt. % to 10 wt. %, preferably about 0.01 wt. % to 5 wt. % based on the total quantity of all non-volatile components of the adhesive system.

All usable active substances have the characteristic that they do not have NCO-reactive functional groups, or that the reaction of any functional groups that may be present with the isocyanate prepolymer is markedly slower compared to the diamine-NCO reaction.

Analgesics which fulfil this requirement are local anaesthetics such as ambucaine, amylocalne, arecaidine, benoxinate, benzocaine, betoxycaine, butacaine, butethamine, bupivacaine, butoxycaine, chloroprocaine, cocaethylene, cocaine, cyclomethycaine, dibucaine, dimethocaine, dimethisoquin, etidocaine, fomocaine, isobutyl p-aminobenzoate, leucinocaine, lidocaine, meperidine, mepivacaine, metabutoxycaine, octacaine, orthocaine, oxethazaine, phenacaine, piperocaine, piridocaine, pramoxine, procaine, procainamide, proparacaine, propoxycaine, pseudococaine, pyrrocaine, ropivacaine, tetracaine, tolycaine, tricaine, trimecaine, tropacocaine, amolanone, cinnamoyl-cocaine, parethoxycaine, propiocaine, myrtecaine and propanocaine.

Opioid analgesics such as morphine and derivatives thereof (e.g. codeine, diamorphine, dihydrocodeine, hydromorphone, oxycodone, hydrocodone, buprenorphine, nalbuphine and pentazocine), pethidine, levomethadone, tilidine and tramadol can also be used.

Likewise, non-steroidal anti-inflammatory drugs (NSAID) such as acetylsalicylic acid, acemetacin, dexketoprofen, diclofenac, aceclofenac, diflunisal, piritramid, etofenamate, felbinac, flurbiprofen, flufenamic acid, ibuprofen, indomethacin, ketoprofen, lonazolac, lornoxicam, mefenamic acid, meloxicam, naproxen, piroxicam, tiaprofenic acid, tenoxicam, phenylbutazone, propyphenazone, phenazone and etoricoxib can be used. Other analgesics such as azapropazone, metamizol, nabumetone, nefopam, oxaceprol, paracetamol and the analgesically active amitriptyline can of course also be used.

As well as the said analgesics, which have an inflammation-inhibiting action, compounds with purely anti-inflammatory activity can also be used. These include the glucocorticoids such as for example cortisone, betamethasone, dexamethasone, hydrocortisone, methylprednisolone, prednisolone, prednisone, budesonide, allotetrahydrocortisone, fludrocortisone, fluprednisolone, fluticasone propionate, etc.

As substances with antiseptic activity, the following compounds inter alia can be used: triclosan (2,4,4′-trichloro-2′ hydroxydiphenyl ether), chlorhexidine and salts thereof, octenidine, chloramphenicol, florfenicol, chlorquinaldol, iodine, povidone-iodine, hexachlorophene, merbromine, PHMB, silver in nanocrystalline form and silver and copper salts.

Furthermore, as substances with antimicrobial activity, antibiotics from the β-lactam (e.g. penicillin and derivatives thereof, cephalosporins), tetracycline (e.g. demeclocycline, doxycycline, oxytetracycline, minocycline, tetracycline), macrolide (e.g. erythromycin, josamycin, spiramycin), lincosamide (e.g. clindamycin, lincomycin), oxazolidinone (e.g. linezolid), gyrase inhibitor (e.g. danofloxacin, difloxacin, enrofloxacin, ibafloxacin, marbofloxacin, nalidixic acid, pefloxacin, fleroxacin, levofloxacin) and cyclic peptide (e.g. bicozamycin) classes can be used. Rifamycin, rifaximin, methenamine; mupirocin, fusidic acid, flumechin, and nitroimidazole (e.g. metronidazole, nimorazole, tinidazole), nitrofuran (furaltadone, nifurpirinol, nihydrazone, nitrofurantoin) and sulphonamide (e.g. sulfabromomethazine, sulfacetamide, sulfachlorpyridazine, sulfadiazine etc.) derivatives and β-lactamase inhibitors such as clavulanic acid can also be used.

As substances with antimycotic activity, all azole derivatives which inhibit the biosynthesis of ergosterol, such as for example clotrimazole, fluconazole, miconazole, bifonazole, econazole, fenticonazole, isoconazole, oxiconazole etc. can be used. Other topically usable antimycotics are amorolfine, ciclopirox, thymol and derivatives thereof and naftifine. The alkylparabens class can also be used.

The compounds with antiparasitic activity include inter alia the ectoparasiticides cyfluthrin and lindane, various azole derivatives such as for example dimetridazole and metronidazole, and quinine.

If necessary, the curing component can be coloured.

The adhesive systems according to the invention are obtained by mixing the prepolymer A with the secondary diamine of the component B). The ratio of amino groups to free NCO groups is preferably 1:1.5 to 1:1, particularly preferably 1:1.

Directly after the mixing together of the individual components, the adhesive systems according to the invention preferably have a shear viscosity at 23° C. of 1000 to 10000 mPas, particularly preferably 2000 to 8000 mPas and quite especially preferably 2500 to 5000 mPas.

The time until curing of the adhesive without tackiness of the surface is attained at 23° C. is typically 30 secs to 10 mins, preferably 1 min to 8 mins, particularly preferably 1 min to 5 mins.

A further subject of the invention are the adhesive films obtainable from the adhesive systems according to the invention and composite parts produced therefrom.

In a preferred embodiment, the adhesive systems according to the invention are used as tissue adhesives for the closure of wounds in human or animal cell associations, so that clamps or suturing for closure can very largely be dispensed with.

The tissue adhesive according to the invention can be used both in vivo and also in vitro, the in vivo use preferably being for example for wound treatment after accidents or operations.

Hence a procedure for the closure or bonding of cell tissues characterized in that the adhesive systems according to the invention are used is also a subject of the present invention.

Further, the use of such adhesive systems for the production of a means for the closure or bonding of cell tissues and the 2-chamber dispensing system necessary for the application comprising the components of the adhesive system essential to the invention is likewise a subject of the invention.

### EXAMPLES

Unless otherwise stated, all percentages given are based on weight.

As a tissue substitute, beef was used. In each case, two pieces of meat (1=4 cm, h=0.3 cm, b=1 cm) were spread with the adhesive at the ends over a 1 cm width and glued overlapping. In each case, the stability of the adhesive layer was tested by pulling.

### Example 1

### Prepolymer A-1

465 g of HDI and 2.35 g of benzoyl chloride were placed beforehand in a 1 l four-necked flask. Within 2 hrs, 931.8 g of a polyether with an ethylene oxide content of 71% and a propylene oxide content of 29% (each based on the total alkylene oxide content) started on TMP (trifunctional) were added at 80° C. and the mixture stirred for 1 hr more. Next, the excess HDI was distilled off by thin film distillation at 130° C. and 0.1 torr. 980 g (71%) of the prepolymer with an NCO content of 2.53% were obtained. The residual monomer content was <0.03% HDI.

### Example 2

### Dimethyl 2,2′-(pentan-1,5-diylbis(azandiyl))bis(4-methylpentanoate))(1)

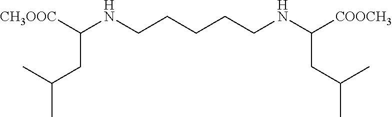

2 mol of Z-protected leucine methyl ester was reacted with 1 mol of glutardialdehyde with stirring for three clays in methanol at room temperature to give the diimine. This was then hydrogenated over Pd/C in methanol. The product was purified by column chromatography.

¹H-NMR (CDCl₃, 400 MHz): δ=0.91 (d, 6H), 0.94 (d, 6H), 1.35 (m, 2H), 1.48 (m, 8H), 1.69 (m, 2H), 2.43 (m, 2H), 2.56 (m, 2H), 3.29 (t, 2H), 3.72 (s, 6H).

¹³C-NMR (CDCl₃, 400 MHz): δ=22.3, 22.5, 24.7, 24.8, 29.9, 42.7, 48.0, 51.4, 59.9, 176.5

### Example 3

### Diethyl 2,2′-(pentan-1,5-diyldiimino)dipropanoate)(2)

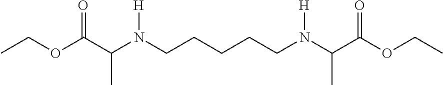

5 g of ethyl pyruvate (2 eq) were dissolved in 100 ml of absolute ethanol and treated with 15.9 g of 1,5-diaminopentane. With ice cooling, 21.2 g (2 eq) of sodium cyanoborohydride were added. The mixture was further stirred overnight at room temperature. After hydrolysis, the product is extracted by shaking with methylene chloride. Purification is then effected by column chromatography (methanol/ethyl acetate 1:6). 3 g of the product were obtained as a yellow liquid.

¹H-NMR (CDCl₃, 400 MHz): δ=1.11 (d, 6H), 1.18 (t, 6H), 1.22 (m, 2H), 1.38 (m, 4H), 2.38 (m, 2H), 2.48 (m, 2H), 3.22 (m, 2H), 4.08 (q, 4H).

¹³C-NMR (CDCl₃, 400 MHz): δ=14.2, 18.8, 24.8, 29.6, 47.7, 56.6, 61.4, 174.6

### Example 4

### Diethyl 2,2′-(butan-1,4-diyldiimino)dipropanoate)(3)

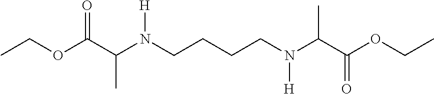

Analogously to Example 2, 3.5 g of the product were obtained as a yellow liquid from 5 g of ethyl pyruvate and 13.7 g of 1,4-diaminobutane.

¹H-NMR (CDCl₃, 400 MHz): δ=1.30 (d, 6H), 1.31 (t, 6H), 1.58 (m, 4H), 2.50 (m, 2H), 2.60 (m, 2H), 3.41 (q, 2H), 4.21 (q, 4H).

¹³C-NMR (CDCl₃, 400 MHz): δ=14.2, 18.4, 27.5, 47.5, 56.6, 60.4, 175.4.

### Example 5

### Dimethyl 2,2′-(propan-1,3-diyldiimino)bis(3-methylbutanoate))(4)

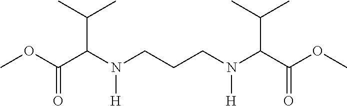

5.36 g of 1,3-dibromopropane (1 eq) were dissolved in 25 ml of methanol and treated with 10.74 g (2 eq) of triethylamine. Next, 8.9 g (1 eq) of l-valine methyl ester hydrochloride were added. The reaction mixture was heated at reflux for five days. After cooling to room temperature, the mixture was taken up in dichloromethane and extracted several times with water. After drying over magnesium sulphate, the solvent was removed under vacuum. 4.2 g of the product were obtained as a yellow liquid.

¹H-NMR (CDCl₃, 400 MHz): δ=0.90 (d, 6H), 0.96 (d, 6H), 1.67 (m, 2H), 1.92 (m, 2H), 2.50 (m, 2H), 2.68 (m, 2H), 2.98 (d, 2H), 3.71 (s, 6H).

¹³C-NMR (CDCl₃, 400 MHz): δ=18.7, 19.2, 19.7, 31.4, 46.6, 51.1, 67.4, 175.4.

### Example 6

### Dimethyl 2,2′-(propan-1,3-diyldiimino)bis(3-methylpentanoate))(5)

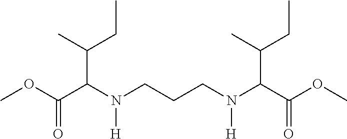

Analogously to Example 4, 4.5 g of the product were produced as a yellow liquid from 5.2 g of 1,3-dibromopropane, 10.43 g of triethylamine and 9.36 g of L-isoleucine methyl ester hydro-chloride.

¹H-NMR (CDCl₃, 400 MHz): δ=0.89 (d, 6H), 0.94 (d, 6H), 1.20 (m, 2H), 1.61 (m, 6H), 2.49 (m, 2H), 2.68 (m, 2H), 3.02 (d, 2H), 3.71 (s, 6H).

¹³C-NMR (CDCl₃, 400 MHz): δ=11.4, 11.7, 15.6, 25.7, 38.4, 47.3, 51.3, 66.2, 175.6.

### Example 7

### Dimethyl 2,2′-(propan-1,3-diyldiimino)bis(3-phenylpropanoate))(6)

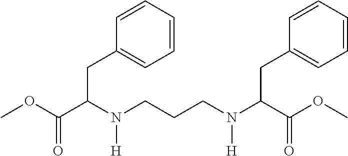

Analogously to Example 4, 3.8 g of the product were obtained from 4.86 g of 1,3-dibromopropane, 9.75 g of triethylamine and 10.39 g of L-phenylalanine methyl ester hydrochloride as a yellow liquid after purification by column chromatography (methanol/ethyl acetate 1:6).

¹H-NMR (CDCl₃, 400 MHz): δ=1.59 (m, 2H), 2.50 (m, 2H), 2.62 (m, 2H), 2.89 (m, 4H), 3.43 (d, 2H), 3.61 (s, 6H), 7.22 (m, 10H).

¹³C-NMR (CDCl₃, 400 MHz): δ=41.1, 46.5, 51.5, 62.8, 63.2, 126.1, 128.3, 129.2, 137.3, 174.9.

### Example 8

### Dimethyl 2,2′-(hexan-1,6-diyldiimino)bis(3-phenylpropanoate))(6)

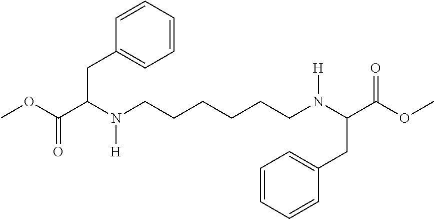

Analogously to Example 4, 3.1 g of the product were obtained from 5.65 g of 1,6-dibromohexane, 9.37 g of triethylamine and 9.99 g of L-phenylalanine methyl ester hydrochloride as a yellow liquid after purification by column chromatography (methanol/ethyl acetate 1:6).

¹H-NMR (CDCl₃, 400 MHz): δ=1.22 (m, 2H), 1.54 (On, 4H), 2.42 (m, 2H), 2.58 (On, 2H), 2.68 (m, 2H), 2.90 (m, 4H), 3.48 (d, 2H), 3.69 (s, 6H), 7.19 (m, 10H).

¹³C-NMR (CDCl₃, 400 MHz): δ=29.9, 36.5, 39.6, 51.4, 63.9, 69.8, 126.0, 128.3, 129.1, 138.8, 174.5.

### Example 9

### Tissue Adhesives

1 g of the prepolymer A-1 were stirred well in a beaker with an equivalent quantity of dimethyl 2,2′-(pentane-1,5-diylbis(azandiyl))bis(4-methylpentanoate). Directly after this, the reaction mixture was applied thinly onto the tissue to be glued. Curing to a transparent film with an associated strong adhesion had taken place within 1 min. After 2 mins, the surface of the adhesive was no longer tacky.

| Compound | Processing time | | Adhesive strength |
|---|---|---|---|
| 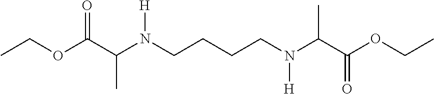 | 42 | secs | − |
| 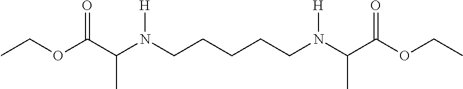 | 33 | secs | − |
| 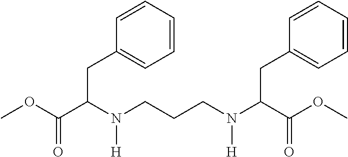 | 4 | mins | − |
| 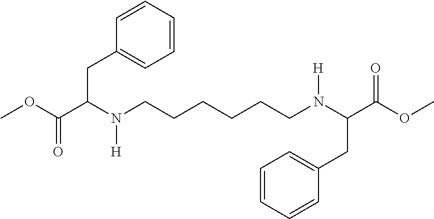 | 4 | mins | − |
| 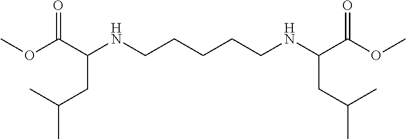 | 4 | mins | ++ |
| 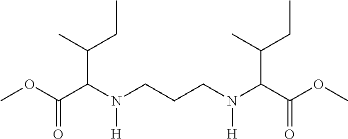 | 3 | mins | ++ |
| 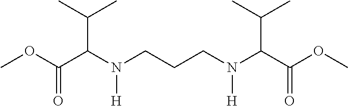 | 4 | mins | ++ |

++: high adhesive strength, the tissue tears on pulling
−: low adhesive strength, the adhesive suture detaches from the tissue

1.-14. (canceled)

15. An adhesive system comprising
A) a prepolymer comprising isocyanate groups obtained from
A1) aliphatic isocyanates and
A2) polyols having a number average molecular weight of greater than or equal to 400 g/mol and an average OH group content of 2 to 6
B1) a secondary diamine of the formula (I) 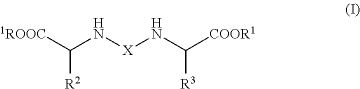
wherein
X represents a divalent, optionally heteroatom-containing, hydrocarbon residue,
R¹ represents, independently of one another, an organic residue which have no Zerevitinov-active hydrogen and
R², R³ represent, independently of one another, an optionally substituted and/or heteroatom-containing hydrocarbon residue having from 1 to 9 carbon atoms or hydrogen,
B2) optionally organic fillers, which exhibit a viscosity measured according to DIN 53019 at 23° C. in the range from 10 to 6000 mPas and
C) optionally one or more pharmacologically active compounds.

16. The adhesive system according to claim 15, wherein the aliphatic isocyanates comprise isocyanate groups which are exclusively aliphatically or cycloaliphatically bound isocyanate groups.

17. The adhesive system according to claim 15, wherein the aliphatic isocyanates have an average NCO group content of 2 to 2.4.

18. The adhesive system according to claim 15, wherein the polyols have a number average molecular weight of 4000 to 8500 g/mol.

19. The adhesive system according to claim 15, wherein the polyols have an average OH group content of 3 to 4.

20. The adhesive system according to claim 15, wherein the polyols comprise polyalkylene oxide polyethers.

21. The adhesive system according to claim 20, wherein the polyalkylene oxide polyethers have a content of ethylene oxide-based units of 60 to 90 mol % based on the total amount of alkylene oxide units.

22. The adhesive system according to claim 15, wherein, in formula (I), X represents an alkyl chain with 4 to 7 carbon atoms; R² and R³ represent, independent of one another, —CH₃, —CH₂CH(CH₃)₂, —CH(CH₃)₂, —C(CH₃)₃, —CH(CH₃)CH₂CH₃, phenyl, 2,3-dihydroxyphenyl, alkyl or cycloalkyl residues having from 1 to 9 carbon atoms, which optionally comprises a heteroatom selected from the group consisting of sulphur, oxygen and nitrogen as part of a functional group in the chain or as a terminal group; and R′ represents a C₁to C₁₀ alkyl residue.

23. The adhesive system according to claim 15, wherein the adhesive system is a tissue adhesive for human or animal tissue.

24. A method for the closure or bonding of cell tissues, comprising applying the adhesive system according to claim 15 to cell tissue.

25. The method according to claim 24, wherein the cell tissue is human or animal tissue.

26. The adhesive system according to claim 15 wherein the adhesive system is used for the production of a composition or device which closes or bonds cell tissues.

27. An Adhesive film or composite part obtained with the adhesive system according to claim 15.

28. A two-chamber dispensing system comprising the adhesive system according to claim 15.
